SEQUENCE LISTING (7 sequences)

SEQ ID NO: 1 - protein, 198 aa, Homo sapiens
MSNVRVSNGSPSLERMDARQAEHPKPSACRNLFGPVDHEELTRDLEKHCRDMEEASQRKWNFDFQNHKPLEGKYEWQEVEKGSLPEFYYRPPRPPKGACKVPAQESQDVSGSRPAAPLIGAPANSEDTHLVDPKTDPSDSQTGLAEQCAGIRKRPATDDSSTQNKRANRTEENVSDGSPNAGSVEQTPKKPGLRRRQT

SEQ ID NO: 2 - DNA, 597 bp, Homo sapiens
atgtcaaacgtgcgagtgtctaacgggagccctagcctggagcggatggacgccaggcaggcggagcaccccaagccctcggcctgcaggaacctcttcggcccggtggaccacgaagagttaacccgggacttggagaagcactgcagagacatggaagaggcgagccagcgcaagtggaatttcgattttcagaatcacaaacccctagagggcaagtacgagtggcaagaggtggagaagggcagcttgcccgagttctactacagacccccgcggccccccaaaggtgcctgcaaggtgccggcgcaggagagccaggatgtcagcgggagccgcccggcggcgcctttaattggggctccggctaactctgaggacacgcatttggtggacccaaagactgatccgtcggacagccagacggggttagcggagcaatgcgcaggaataaggaagcgacctgcaaccgacgattcttctactcaaaacaaaagagccaacagaacagaagaaaatgtttcagacggttccccaaatgccggttctgtggagcagacgcccaagaagcctggcctcagaagacgtcaaacgtaa

SEQ ID NO: 3 - DNA, 20 bp, Homo sapiens
tggagggcagatacgaatgg

SEQ ID NO: 4 - DNA, 20 bp, Homo sapiens
ggggaaccgtctgaaacatt

SEQ ID NO: 5 - DNA, 20 bp, Homo sapiens
gggacgcggcgctcggtcat

SEQ ID NO: 6 - RNA, 22 nt, Homo sapiens
aagucucaucggauuuugcagc

SEQ ID NO: 7 - RNA, 21 nt, Artificial Sequence
  scrambled control
cagcgcugacaacaguuucau

(19) United States
(12) Patent Application Publication
(10) Pub. No.: US 2013/0150429 A1
(43) Pub. Date: Jun. 13, 2013

(54) P27KIP1 AS A MOLECULAR MARKER FOR SUITABILITY AND EFFICACY OF TREATMENT WITH HSP27 INHIBITORS

(71) Applicant: The University of British Columbia, Vancouver (CA)

(72) Inventors: Martin E. Gleave, Vancouver (CA); Christopher Ong, Vancouver (CA)

(73) Assignee: THE UNIVERSITY OF BRITISH COLUMBIA, Vancouver (CA)

(21) Appl. No.: 13/707,061

(22) Filed: Dec. 6, 2012

Related U.S. Application Data
(60) Provisional application No. 61/568,856, filed on Dec. 9, 2011.

(51) Int. Cl. G01N 33/68; C12Q 1/68

(52) U.S. Cl. CPC G01N 33/6893; C12Q 1/6886
USPC 514/44 A; 435/7.1; 435/7.92; 435/6.11; 514/280; 514/733; 514/682; 514/257; 514/279

(57) ABSTRACT

Cells expressingHsp27 exhibit reduced levels of p27kip1. Accordingly, a method for treatment of cancer using hsp27 inhibition that includes a preliminary test to ascertain the status of the p27kip1 in the target cells. In this test, a sample of cancerous tissue from the patient from the patient (including a human patient) and evaluated to determine an expression of level of functional p27kip1. In the case where the expression level of p27kip1 is below a threshold level, a therapeutic composition comprising as an active agent a composition effective to inhibit the expression or activity of hsp27 in administered to the patient.

### STATEMENT OF RELATED CASES

This application claims the benefit of U.S. Provisional Application No. 61/568,856 filed Dec. 9, 2011, which application is incorporated herein by reference in its entirety.

### FIELD OF THE INVENTION

Hsp27 inhibitors have been disclosed for use in the treatment of cancer. This application relates to molecular markers which are predictive of the efficacy of such inhibitors, and to the use of methods assessing the amounts of such markers in the in assessing tumor susceptibility to anti-HSP27 therapy. In particular, the marker used in present invention is p27kip1. This marker can also be used to assess the effectiveness of on-going treatment with an Hsp27 inhibitors.

### BACKGROUND OF THE INVENTION

Heat shock protein 27 (Hsp27) is a 27-kDa molecular chaperone protein that is induced and phospho-activated in response to a variety of cytotoxic stressors, including hormonal, chemo- and radiotherapy. (Garrido C, Brunet M, Didelot C, Zermati Y, Schmitt E, Kroemer G. Heat shock proteins 27 and 70: anti-apoptotic proteins with tumorigenic properties. Cell Cycle 2006; 5: 2592-2601). Increased expression of Hsp27 correlates with suppression of apoptosis and improved survival from a variety of cytotoxic insults. In cancer, Hsp27 is frequently overexpressed in numerous malignancies. (Ciocca DR, Calderwood SK. Heat shock proteins in cancer: diagnostic, prognostic, predictive, and treatment implications. Cell Stress Chaperones 2005; 10: 86-103. 3. Rocchi P, So A, Kojima S, Signaevsky M, Beraldi E, Fazli L et al. Heat shock protein 27 increases after androgen ablation and plays a cytoprotective role in hormone-refractory prostate cancer. Cancer Res 2004; 64: 6595-6602.) Elevated expression of Hsp27 in cancer has been associated with poor clinical prognosis and therapeutic resistance. Overexpression of Hsp27 in bladder, colon and prostate cancer cells enhances tumor growth and progression in vivo. (Rocchi P, Beraldi E, Ettinger S, Fazli L, Vessella R L, Nelson C et al. Increased Hsp27 after androgen ablation facilitates androgen-independent progression in prostate cancer via signal transducers and activators of transcription 3-mediated suppression of apoptosis. Cancer Res 2005; 65: 11083-11093; Kamada M, So A, Muramaki M, Rocchi P, Beraldi E, Gleave M. Hsp27 knockdown using nucleotide-based therapies inhibit tumor growth and enhance chemotherapy in human bladder cancer cells. Mol Cancer Ther 2007; 6: 299-308; Garrido C, Fromentin A, Bonnotte B, Favre N, Moutet M, Arrigo A P et al. Heat shock protein 27 enhances the tumorigenicity of immunogenic rat colon carcinoma cell clones. Cancer Res 1998; 58: 5495-5499.) Furthermore, selective inhibition of Hsp27 expression with antisense oligonucleotide (ASO)-based therapy has been shown to suppress tumor growth and sensitize cancer cells to hormonal, chemo- and radiotherapy. On the basis of these preclinical proof-of-principle studies, OGX-427 (Oncogenex), a selective, second-generation antisense oligonucleotide (ASO) inhibitor of Hsp27, has recently advanced into phase I/II clinical trials for treatment of a variety of cancers.

Although Hsp27 inhibitors such as OGX-427 show promise in the treatment of cancer, it is the case with these inhibitors, as with other cancer therapies, that they are not universally successful. For example, US 2009-0281166 A1, which is incorporated herein by reference, discloses the use of a preliminary test for the amount of phosphatase and tensin homologue deleted from chromosome 10 (PTEN) as a basis for selecting candidate individuals for treatment with an Hsp27 inhibitor.

### SUMMARY OF THE INVENTION

The present inventors have now found that the amount of the protein referred to as p27kip1 in target cancer cells is a predictive indicator of the activity of Hsp27 inhibitors as a therapeutic. Specifically, as demonstrated below, cells expressingHsp27 exhibit reduced levels of p27kip1. Accordingly, the present invention provides a method for the treatment of cancer using hsp27 inhibition that includes a preliminary test to ascertain the status of the p27kip1 in the target cells.

In accordance with the present invention there is provided a method for treating cancer in a patient diagnosed as suffering from cancer comprising the steps of:

(a) obtaining a sample of cancerous tissue from the patient;

(b) evaluating the sample of cancerous tissue to determine an expression of level of functional p27kip1; and

(c) in the case where the expression level of p27kip1 is below a threshold level, administering to the patient a therapeutic composition comprising as an active agent a composition effective to inhibit the expression or activity of hsp27. The nature of the active agent is not critical, although in certain specific embodiments, the active agent is an antisense oligonucleotide (such as ONGX-427, Oncogenex) or a duplex siRNA.

The invention further provides a method for assessing the susceptibility of a tumor in an animal (such as man) to treatment with an anti-HSP27 drug by assessing the p27kip1 level in said tumor, and comparing that level with the level of p27kip1 in surrounding normal tissue or a reference level.

The invention further provides a method for monitoring the affect of an anti-hsp27 drug on the tumor of an animal (such as man), the method comprising the steps of measuring the level of p27kip1 before and after treatment of said animal to determine whether an increase of p27kip1 has occurred. Observation of an increase in p27kip1 is indicative that Hsp27 reduction is occurring and therefore that the therapy is progressing as expected.

### BRIEF DESCRIPTION OF THE DRAWINGS

FIG. 1 shows cell growth rates of LNCaP_{Hsp27} and LNCaP_{mock} as observed by direct counting of viable cells.

FIG. 2 shows results for cell proliferation of LNCaP_{mock} and LNCaP_{Hsp27} cells assessed by [³H]-thymidine incorporation into DNA. The symbol ‘*’ denotes statistical significance (P>0.05).

FIG. 3 shows cell numbers of LnCap cells treated with anti-Hsp27 siRNA.

### DETAILED DESCRIPTION OF THE INVENTION

The present invention provides methods relating to the treatment of cancer in a patient diagnosed as suffering from cancer. In preferred embodiments, the patient is a human patient, although the method can also be used in veterinary applications, for example in the treatment of cancer in dogs, cats and other pets or agricultural animals.

As used in the specification and claims of the present application, the term “treating” refers to performing the method steps of the invention with intention and expectation of a therapeutic benefit to the patient. It would be understood in the art that not all patients respond favorably, or to the same extent to a given treatment. Furthermore, it will be understood in the art that the results of obtained for any individual cannot be compared to results for that individual in the absence of the treatment. Thus, actual therapeutic benefit is not required to fall within the scope of the concept of “treating.”

The occurrence of elevated levels of Hsp27 in various types of cancer and the demonstrated efficacy of Hsp27 inhibitors in multiple types of cancers is indicative of the general applicability of the present invention to cancers of many types. In general, the method will be employed with cancer types which are considered to be targets for Hsp27 therapy, including in particular those where there has been a previous determination of Hsp27 overexpression for the patient's cancer. Specific non-limiting examples of cancer types that may be treated using the method of the invention include breast, prostate, ovarian, uterine, non-small cell lung, bladder, gastric, liver, endometrial, laryngeal and colorectal cancers; squamous cell carcinomas such as esophageal squamous cell carcinoma, glioma, glioblastoma, melanoma, multiple myelmoma and lymphoma.

The first step of the present method is obtaining a sample of cancerous tissue from the patient for evaluation. Such samples can be obtained using normal biopsy and sampling techniques consistent with the type of cancer. The size of the sample needed is based upon the evaluation procedure to be employed.

Once a sample of cancerous tissue is obtained it is evaluated to determine an expression of level of p27kip1. As used herein, the term “p27kip1” refers to cyclin-dependent kinase inhibitor p27. The sequence of this protein in humans is known from Accession No. AAA20240 as

| Seq ID No. 1 | |
|---|---|
| 1 | msnvrvsngs pslermdarq aehpkpsacr nlfgpvdhee ltrdlekhcr dmeeasqrkw |
| 61 | nfdfqnhkpl egkyewqeve kgslpefyyr pprppkgack vpaqesqdvs gsrpaaplig |
| 121 | apansedthl vdpktdpsds qtglaeqcag irkrpatdds stqnkranrt eenvsdgspn |
| 181 | agsveqtpkk pglrrrqt. |

The corresponding mRNA sequence (Accession No. U10906) is:

| Seq ID No. 2 | |
|---|---|
| 1 | atgtcaaacg tgcgagtgtc taacgggagc cctagcctgg agcggatgga cgccaggcag |
| 61 | gcggagcacc ccaagccctc ggcctgcagg aacctcttcg gcccggtgga ccacgaagag |
| 121 | ttaacccggg acttggagaa gcactgcaga gacatggaag aggcgagcca gcgcaagtgg |
| 181 | aatttcgatt ttcagaatca caaaccccta gagggcaagt acgagtggca agaggtggag |
| 241 | aagggcagct tgcccgagtt ctactacaga cccccgcggc cccccaaagg tgcctgcaag |
| 301 | gtgccggcgc aggagagcca ggatgtcagc gggagccgcc cggcggcgcc tttaattggg |
| 361 | gctccggcta actctgagga cacgcatttg gtggacccaa agactgatcc gtcggacagc |
| 421 | cagacggggt tagcggagca atgcgcagga ataaggaagc gacctgcaac cgacgattct |
| 481 | tctactcaaa acaaaagagc caacagaaca gaagaaaatg tttcagacgg ttccccaaat |
| 541 | gccggttctg tggagcagac gcccaagaag cctggcctca gaagacgtca aacgtaa. |

There are numerous methods by which the level of functional p27kip1 may be determined including immunohistochemical methods, p27kip1 specific immunoassays such as ELISA and methods based on detection of the p27kip1 mRNA.

For immunoassays, anti-p27kip1 antibodies are available from a variety of commercial suppliers, including Abcam, Cell Signaling Technology and Novus Biologicals for use in a immunoassays of different types including Western Blots, ELISA assays, and the like as described for example in Chang, S.F. et al. (2008) Proc Natl Acad Sci U S A 105, 3927-2 and Zhang, S. et al. (2009) Mol Cancer Res 7, 570-80.

Detection of p27kip1 by Northern Blot analysis is described in art, for example in Park et al., EMBO reports (2008) 9, 766-773. RT-PCR analysis of p27kip1 has been described using the following primers: upstream sequence, 5′-TGGAGGGCAGATACGAATGG-3′ (Seq ID No. 3); downstream sequence, 5′-GGGGAACCGTCTGAAACATT-3′ (Seq ID No. 4) which should yield a 327-bp product. (Joyce et al. Invest. Ophthalmol. Vis. Sci. July 2002 vol. 43 no. 7 2152-2159.)

The test result of the performed assay is compared to a relevant threshold level. The relevant threshold level is determined for the tissue type tested and for the assay performed. When a standard level is used in the comparison, the threshold level reflects an average or lower than average amount of p27kip1 in normal (i.e. non-cancerous) samples of the same tissue type. In the alternative, the threshold value may be the amount of p27kip1 in adjacent normal cells from the same individual based on a side-by-side test.

It will be appreciated that the selection of a specific numerical threshold value is a balance between the likelihood of missing the opportunity to give appropriate therapy to a patient with a higher, but still reduced level of p27kip1 against the risk of treating a patient with a therapeutic that will not be effective resulting in a delay in administering alternative therapy. Thus, the specific threshold selected for any given cancer will depend on the variability of p27kip1 levels in non-cancerous “normal” tissues, the precision and accuracy of the assay employed, and the availability of viable alternative treatment modalities for the cancer type.

When the assay reveals an amount of p27kip1 that is below the threshold level, a therapeutic composition comprising as an active agent a composition effective to inhibit Hsp27 is administered to the patient. Inhibitors of Hsp27 expression of various different types are known in the art. The specific route of administration, the dosage level and the treatment frequency will depend on the nature of the active agent employed. In general, the therapeutic agent may be administered by intravenous, intraperitoneal, subcutaneous, topical or oral routes, or direct local tumor injection. For example, antisense targeting hsp27 (such as gggacgcggc gctcggtcat, OGX-427, SEQ ID No. 5) may be administered at levels of injection at 200 mg, 400 mg, 600 mg, 800 mg or 1000 mg once a week as tolerated by the patient.

U.S. Pat. No. 7,101,991 discloses antisense oligonucleotides and siRNA that inhibit hsp27 expression. Additional oligonucleotide sequences targeting hsp27 expression are disclosed in WO2007/025229. Non-oligonucleotide compounds for inhibition of hsp27 have been disclosed, including berberine derivatives described in European Patent EP0813872, and compounds described in JP 10045572, JP 10045574, JP10036261 and JP 10036267, all assigned to Kureha Chemical Industries Co,. Ltd. Paclitaxel has also been shown to be an inhibitor of hsp27 expression. Tanaka et al., Int J Gynecol Cancer. 2004 July-August;14(4):616-20. Other inhibitors include magnolol-containing synthetic suppressors of protein belonging to hsp27 family, shikonin-containing synthetic suppressors of protein belonging to hsp27 family and aconitine-containing synthesis inhibitors of protein belonging to hsp27 family.

The invention further provides a method for assessing the susceptibility of a tumor in an animal (such as man) to treatment with an anti-HSP27 drug by assessing the p27kip1 level in said tumor, and comparing that level with the level of p27kip1 in surrounding normal tissue or a reference level. In this case, the result of the test performed as discussed above is used in the selection of a therapeutic, i.e. the decision whether or not to employ an anti-Hsp27 therapeutic.

The invention further provides a method for monitoring the affect of an anti-hsp27 drug on the tumor of an animal (such as man), the method comprising the steps of measuring the level of p27kip1 before and after treatment of said animal to determine whether an increase of p27kip1 has occurred. Observation of an increase in p27kip1 is indicative that Hsp27 reduction is occurring and therefore that the therapy is progressing as expected.

Having described the invention above, the following non-limiting examples are provided to further illustrate and demonstrate the invention. These experiments show that LNCaP_{Hsp27} cells, a prostate cancer cell line in which expresses Hsp27 exhibited increased levels of cyclin D1 and CDK2, with a concomitant decrease in cyclin-dependent kinase inhibitor p27 (p27kip1) expression. Thus, p27kip1 serves as an alternative indicator of the

### EXAMPLE

In the following example, the materials and methods used were as follows:

Cell Lines and Materials.

LNCaP cells were purchased from American Type Culture Collection (Rockville, Md., USA). LNCaP cells (used up to passage 50 in the present study), were routinely maintained in RPMI1640 (Life Technologies, Burlington, ON, Canada).

Antibodies against Hsp27, phospho-Hsp27 (Ser-82) (StressGen, Victoria, BC, USA), PEA-15 (Santa Cruz Biotechnology, Santa Cruz, Calif., USA), phospho-PEA-15 (Ser-116; Biosource, Burlington, ON, Canada), Akt, phospho-Akt (Ser-473), phospho-Foxo-1 (Ser-256; Cell Signaling Technology, Danvers, Mass., USA), FADD (Upstate), p27kip1, cyclin D1, CDK2 (Santa Cruz Biotechnology) and vinculin (Sigma-Aldrich) were used according to manufacturer's instructions.

Lentiviral Transduction of LNCaP Cells.

Two vectors, pHRO-cytomegalovirus (CMV)-Hsp27 and pHRO-CMV as an empty vector, were used in the present study as previously described. (Rocchi et al, 2005, supra) pHRO-CMV-Hsp27 contains the full-length cDNA for human Hsp27 subcloned into the BamHI and XhoI sites of the pHRO-CMV-enhanced green fluorescent protein (GFP) lentiviral vector. Lentiviruses were prepared and transduced into LNCaP. Expression of GFP and Hsp27 in transduced LNCaP cells was verified by fluorescence microscopy and western blotting, respectively. All constructs were confirmed by DNA sequence analysis.

siRNA-Mediated Gene Silencing.

LNCaP cells were plated at 7×10⁵ per 10 cm dish. After 24 h, cells were transfected with siHsp27 or Scr duplexes as previously described.27 Briefly, the RNA duplex was diluted in Opti-MEM I serum-free medium and Oligofectamin (Invitrogen) and incubated at room temperature for 20 min before addition to cells. The media was changed after 4-6 h. Cells were used 48 h after transfection. The following siRNAs were used: siAkt (Cell Signaling Technology), siHsp27 (5′-AAGUCUCAUCGGAUUUUGCAGC-3′ (Seq ID No. 6)); Dharmacon, Lafayette, Colo., USA) and Scr (5′-CAGCGCUGACAACAGUUUCAU-3′ (Seq ID No 7)).

Cell Proliferation Assay.

Cells were seeded at 0.5×10⁵ cells per well in six-well dishes. At 24 h after culture, cell growth was quantified by direct cell counting at 2-day intervals up to 7 days. Each experiment was repeated three times. Alternatively, cell number was quantified using the Invitrogen CyQuant cell proliferation assay as per the manufacturer's protocols.

[³H]-Thymidine Incorporation Assay.

Cells were seeded at 4×10⁴ cells/ml in 12-well plates in normal growth media for 24 h. At 24 or 48 h after culture, 10 ml of 100 μCi/ml [³H]-thymidine was added per well and cells were incubated for 3 h. The cells were detached from the plate with a trypsin-EDTA solution (0.05% trypsin and 0.53 mM EDTA; Life Technologies Inc., Gaithersburg, Md., USA). After centrifuging, cells were resuspended in 100 ml ddH2O and were transferred to 96-well plates. The collected cells were harvested onto glass-fiber filter mats using a Tomtec Harvester 96 Mach 3M (Hamden, Conn., USA) and counted on a Wallac 1450 Microbeta plate scintillation counter (Turku, Finland). Each experiment was performed six times

Example.

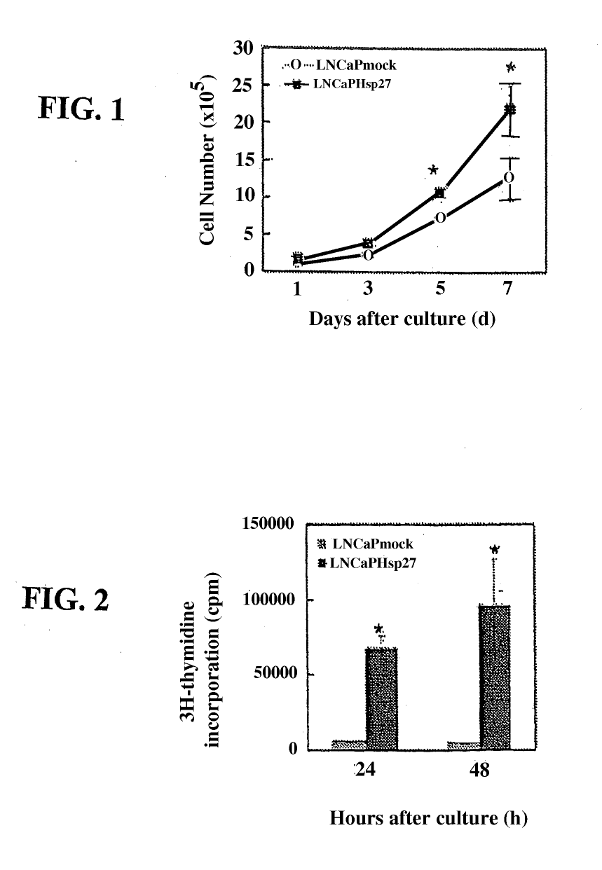

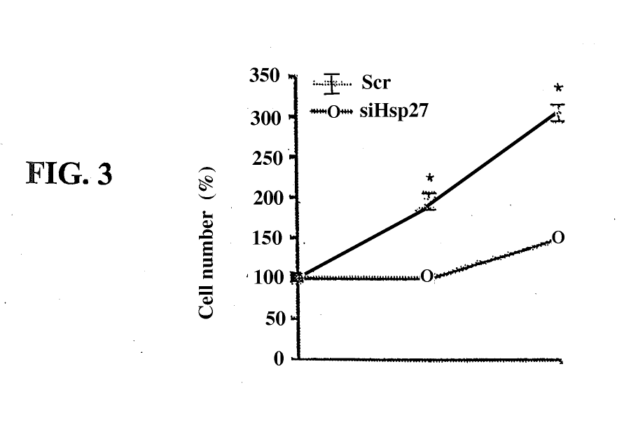

The observation that Hsp27 overexpression leads to enhanced tumor growth and progression raises intriguing questions regarding whether Hsp27 might also be able to confer growth-promoting properties that contribute to the process of tumorigenesis, in addition to its cytoprotective functions. To further examine the effects of Hsp27 on cell growth, LNCaP cells were transduced with a lentiviral vector encoding an Hsp27 expression cassette (LNCaP_{H5p27}) or an empty vector (LNCaP_{mock}) as a control. As shown in FIG. 1, LNCaP_{H5p27} cells expressed increased levels of Hsp27 protein that is primarily found in its phosphorylated, activated form. Consistent with previous findings, LNCaP_{H5p27} cells exhibited enhanced cell growth as compared with LNCaP_{mock} (FIG. 1). To investigate whether Hsp27 improves cell growth, in part, by promoting cell cycle progression, cell proliferation of LNCaP_{mock} versus LNCaP_{Hsp27} cells was analyzed by assessing [³H]-thymidine incorporation as a measure of DNA synthesis and by immunoblotting for panel of typical cell cycle markers. A greater extent of thymidine incorporation was observed in LNCaP_{Hsp27} than in LNCaP_{mock} cells, indicating that Hsp27 overexpression leads to an increased proportion of cells undergoing DNA synthesis (FIG. 2). LNCaP_{H5p27} cells also expressed increased levels of cyclin D1 and CDK2, with a concomitant decrease in cyclin-dependent kinase inhibitor p27 (p27kip1) expression. Conversely, small interfering RNA (siRNA)-mediated silencing of Hsp27 inhibits LNCaP cell growth (FIG. 3).

All of the publications and patents cited herein are incorporated herein by reference.

1. A method for treating cancer in a patient diagnosed as suffering from cancer comprising the steps of:
(a) obtaining a sample of cancerous tissue from the patient;
(b) evaluating the sample of cancerous tissue to determine an expression of level of functional p27kip1; and
(c) in the case where the expression level of p27kip1 is below a threshold level, administering to the patient a therapeutic composition comprising as an active agent a composition effective to inhibit the expression or activity of hsp27.

2. The method of claim 1, wherein the active agent is an oligonucleotide that interacts with cellular nucleic acids encoding hsp27 in a sequence specific manner.

3. The method of claim 2, wherein the oligonucleotide is an antisense oligonucleotide.

4. The method of claim 3, wherein the antisense oligonucleotide comprises SEQ ID NO. 5.

5. The method of claim 2, wherein the oligonucleotide is an siRNA oligonucleotide.

6. The method of claim 5, wherein the siRNA oligonucleotide comprises SEQ ID NO. 6.

7. The method of claim 1, wherein the active agent comprises a berberine derivative.

8. The method of claim 1, wherein the active agent comprises a material selected from the group consisting of magnolol-containing synthetic suppressors of protein belonging to hsp27 family, shikonin-containing synthetic suppressors of protein belonging to hsp27 family and aconitine-containing synthesis inhibitors of protein belonging to hsp27 family.

9. The method of claims 1, wherein the active agent comprises evodiamine.

10. The method of claim 1, wherein the step of evaluating the sample is performed using an antibody specific for p27kip1.

11. The method of claims 10, wherein the active agent is an oligonucleotide that interacts with cellular nucleic acids encoding hsp27 in a sequence specific manner.

12. The method of claim 11, wherein the oligonucleotide is an antisense oligonucleotide.

13. The method of claim 12, wherein the antisense oligonucleotide comprises SEQ ID NO. 5.

14. The method of claim 11, wherein the oligonucleotide is an siRNA oligonucleotide.

15. The method of claim 14, wherein the siRNA oligonucleotide comprises SEQ ID NO. 6.

16. The method of 10, wherein the active agent comprises a berberine derivative.

17. The method of claim 10, wherein the active agent comprises a material selected from the group consisting of magnolol-containing synthetic suppressors of protein belonging to hsp27 family, shikonin-containing synthetic suppressors of protein belonging to hsp27 family and aconitine-containing synthesis inhibitors of protein belonging to hsp27 family.

18. The method of claim 10, wherein the active agent comprises evodiamine.

19. The method of claim 1, wherein the step of evaluating the sample is performed using nucleotide analysis techniques.

20. The method of claim 19, wherein the active agent is an oligonucleotide that interacts with cellular nucleic acids encoding hsp27 in a sequence specific manner.

21. The method of claim 20, wherein the oligonucleotide is an antisense oligonucleotide.

22. The method of claim 21, wherein the antisense oligonucleotide comprises SEQ ID NO. 5.

23. The method of claim 20, wherein the oligonucleotide is an siRNA oligonucleotide.

24. The method of claim 23, wherein the siRNA oligonucleotide comprises SEQ ID NO. 6.

25. The method of claim 19 , wherein the active agent comprises a berberine derivative.

26. The method of claim 19, wherein the active agent comprises a material selected from the group consisting of magnolol-containing synthetic suppressors of protein belonging to hsp27 family, shikonin-containing synthetic suppressors of protein belonging to hsp27 family and aconitine-containing synthesis inhibitors of protein belonging to hsp27 family.

27. The method of claim 19, wherein the active agent comprises evodiamine.

28. The method of claim 1, wherein the patient is human.

29. A method for assessing the susceptibility of a tumor in an animal to treatment with an anti-HSP27 drug by evaluating the p27kip1 level in said tumor, and comparing that level with the level of p27kip1 in surrounding normal tissue or a reference level.

30. A method for monitoring the affect of an anti-hsp27 drug on the tumor of an animal (such as man), the method comprising the steps of evaluating the level of p27kip1 before and after treatment of said animal to determine whether an increase of p27kip1 has occurred, wherein observation of an increase in p27kip1 is indicative that Hsp27 reduction is occurring.
